(19) United States
(12) Patent Application Publication
(10) Pub. No.: US 2026/0130588 A1
(43) Pub. Date: May 14, 2026

(54) IRIS RECOGNITION APPARATUS AND METHOD FOR CORRECTING PUPIL DEVIATION ATTRIBUTABLE TO DOMINANT EYE

(71) Applicant: AJ2 Co., Ltd., Seongnam-si (KR)

(72) Inventors: Gil-Soo Jung, Ansan-si (KR); You-Jung Kim, Ansan-si (KR); Kisuk Jang, Seongnam-si (KR)

(73) Assignee: AJ2 Co., Ltd., Seongnam-si (KR)

(21) Appl. No.: 19/381,970

(22) Filed: Nov. 6, 2025

(30) Foreign Application Priority Data
Nov. 11, 2024 (KR) 10-2024-0159325

(51) Int. Cl. A61B 3/12; A61B 3/113; A61B 3/14

(52) U.S. Cl. CPC A61B 3/1216; A61B 3/113; A61B 3/14

(57) ABSTRACT

Disclosed herein are an iris recognition apparatus and method for correcting pupil deviation attributable to a dominant eye. The iris recognition apparatus includes an iris recognition unit configured to recognize specular reflections generated on a face of a user, pupil positions of both eyes, and iris recognition positions using a camera module, and to determine whether each of the pupil positions is aligned with a position of a corresponding specular reflection, an indicator control unit configured to generate specular reflections at the iris recognition positions by activating an iris recognition IR-LED indicator, and to activate an additional indicator arranged on a side of an eyeball on which a pupil position is misaligned with a position of a specular reflection, and an iris image generation unit configured to capture iris images of both eyes in a state in which the specular reflections are aligned with pupil positions of both eyes.

### CROSS REFERENCE TO RELATED APPLICATION

This application claims the benefit of Korean Patent Application No. 10-2024-0159325, filed November 11, 2024, which is hereby incorporated by reference in its entirety into this application.

### BACKGROUND OF THE INVENTION

### 1. Technical Field

The present disclosure relates generally to an iris recognition technology, and more particularly to an iris recognition technology for correcting pupil deviation (displacement) attributable to a dominant eye.

### 2. Description of the Related Art

A dominant eye (also referred to as a leading or preferred eye) is the one that provides slightly stronger input to the brain and is more relied on for accurate visual tasks such as aiming at or focusing on distant objects. Just as most people have a dominant hand, eyes also tend to exhibit dominance. However, such dominance is generally less noticeable in daily (everyday) activities. When a supplementary description is made, the dominant eye is the eye preferred by the brain to perform visual processing. When both eyes work together to form a single image, one eye tends to take the lead.

When a medical basis for a dominant eye (also referred to as a leading eye) is examined, there is medical evidence supporting the existence of the dominant eye (or the leading eye), as substantiated by research in fields such as ophthalmology, neurology, and psychology. The dominance of eyes is a well-documented phenomenon, and it is recognized that, especially in tasks requiring concentration and precision, one eye plays a more significant role in visual processing than the other eye.

When a neurological perspective is considered, in terms of cerebral hemisphere specialization, the brain processes visual information from both eyes, but typically, one eye sends a stronger signal to the dominant cerebral hemisphere, thus leading the brain to favor the corresponding eye. This dominance is associated with a method in which the brain integrates binocular vision (vision using both eyes) to form a single image. In terms of visual cortical asymmetry, research using neuroimaging techniques such as functional MRI (fMRI) has shown that the visual cortex may exhibit asymmetry in the way it processes information from each eye. The dominant eye often exerts a greater influence on perception due to this cortical preference.

In an iris recognition apparatus without guide bars that block the surrounding environment/background, issues related to dominant and non-dominant eyes may arise. In addition, even when guide bars that block the surrounding environment or background are present as in the case of goggles within approximately 15 cm, distortion of iris images may still occur due to the problem of dominant/non-dominant eyes. Further, when iris recognition is used as payment means in financial markets, a user with a serious disease of the dominant eye (or leading eye) may undergo problems in terms of iris image quality, and thus there is a need to solve these problems.

Meanwhile, Korean Patent No. 10-1797948 entitled “Apparatus for guiding user and method for using the same” discloses a user guidance apparatus and method that acquire an image of a user using a camera, emit illumination light to an eye region of a left or right eye, and adjust the focal position of the camera based on the user’s image, thus guiding the user.

### SUMMARY OF THE INVENTION

Accordingly, the present disclosure has been made keeping in mind the above problems occurring in the prior art, and an object of the present disclosure is to avoid a phenomenon in which an iris image of a non-dominant eye is distorted due to pupil deviation (displacement) attributable to dominance of a dominant eye (leading eye).

Another object of the present disclosure is to capture accurate images of an iris at all angles by solving a phenomenon in which a specular reflection does not occur at an accurate iris recognition position due to pupil deviation (displacement).

In accordance with an aspect of the present disclosure to accomplish the above objects, there is provided an iris recognition apparatus for correcting pupil deviation attributable to a dominant eye, including an iris recognition unit configured to recognize specular reflections generated on a face of a user, pupil positions of both eyes, and iris recognition positions using a camera module, and to determine whether each of the pupil positions is aligned with a position of a corresponding specular reflection, an indicator control unit configured to generate the specular reflections at the iris recognition positions by activating an iris recognition infrared light-emitting diode (IR-LED) indicator, and to activate an additional indicator arranged on a side of an eyeball on which a pupil position is misaligned with a position of a specular reflection, and an iris image generation unit configured to capture iris images of both of the eyes in a state in which the specular reflections are aligned with the pupil positions of both of the eyes.

The iris recognition unit may be configured to, when a center position of the corresponding specular reflection deviates from a center of the corresponding pupil position by a preset threshold distance or longer, determine that the pupil position is misaligned with the position of the corresponding specular reflection.

The iris recognition apparatus may further include a half mirror configured to guide the pupil positions of both of the eyes to the iris recognition positions by reflecting pupils of the user.

The additional indicator may be arranged within 2 cm from each iris recognition position reflected in the half mirror.

The additional indicator may include a visible-light LED additional indicator and an IR-LED additional indicator, the visible-light LED additional indicator may be arranged above the half mirror, and the IR-LED additional indicator may be arranged on a rear surface of the half mirror.

In accordance with another aspect of the present disclosure to accomplish the above objects, there is provided an iris recognition method for correcting pupil deviation attributable to a dominant eye, performed by an iris recognition apparatus for correcting pupil deviation attributable to a dominant eye, the iris recognition method including recognizing a face of a user and pupil positions of both eyes using a camera module, generating specular reflections at iris recognition positions by activating an iris recognition IR-LED indicator, recognizing the specular reflections generated at the iris recognition positions, determining whether each of the pupil positions is aligned with a position of a corresponding specular reflection, when it is determined that each of the pupil positions is misaligned with the position of the corresponding specular reflection, activating an additional indicator arranged on a side of an eyeball on which the pupil position is misaligned with the position of the corresponding specular reflection, and capturing iris images of both of the eyes in a state in which the specular reflections are aligned with the pupil positions of both of the eyes.

The determining may include, when a center position of the corresponding specular reflection deviates from a center of the corresponding pupil position by a preset threshold distance or longer, determining that the pupil position is misaligned with the position of the corresponding specular reflection.

The recognizing may include guiding the pupil positions of both of the eyes to the iris recognition positions by reflecting pupils of the user by using a half mirror.

The additional indicator may be arranged within 2 cm from each iris recognition position reflected in the half mirror.

The additional indicator may include a visible-light LED additional indicator and an IR-LED additional indicator, the visible-light LED additional indicator may be arranged above the half mirror, and the IR-LED additional indicator may be arranged on a rear surface of the half mirror.

### BRIEF DESCRIPTION OF THE DRAWINGS

The above and other objects, features and advantages of the present disclosure will be more clearly understood from the following detailed description taken in conjunction with the accompanying drawings, in which:

FIG. 1 is a diagram illustrating a situation in which an iris recognition error caused by pupil deviation of a non-dominant eye relative to a dominant eye occurs according to an embodiment of the present disclosure;

FIG. 2 is a diagram illustrating a goggle-type iris recognition apparatus according to an embodiment of the present disclosure;

FIG. 3 is a diagram illustrating a dominant eye correction process according to an embodiment of the present disclosure;

FIG. 4 is a block diagram illustrating an iris recognition apparatus for correcting pupil deviation attributable to a dominant eye according to an embodiment of the present disclosure;

FIG. 5 is an operation flowchart illustrating an iris recognition method for correcting pupil deviation attributable to a dominant eye according to an embodiment of the present disclosure; and

FIG. 6 is a diagram illustrating a computer system according to an embodiment of the present disclosure.

### DESCRIPTION OF THE PREFERRED EMBODIMENTS

The present disclosure will be described in detail with reference to the attached drawings. Repeated descriptions and descriptions of known functions and configurations which have been deemed to make the gist of the present disclosure unnecessarily obscure will be omitted below. The embodiments of the present disclosure are provided to more fully describe the present disclosure to those skilled in the art. Therefore, the shapes, sizes, etc. of elements in the drawings may be exaggerated for clear illustration.

In the entire specification, when a certain element is described as “comprising” or “including” a specific component, it means that, unless explicitly stated otherwise, the certain element may further include additional components without excluding the additional components.

The present disclosure may be variously modified and may have various embodiments, and the embodiments are intended to be illustrated and described in detail in the accompanying drawings.

However, this is not intended to limit the present disclosure to particular embodiments, and it should be appreciated that all changes, equivalents, and substitutes that do not depart from the spirit and technical scope of the present disclosure are encompassed in the present disclosure.

In description of components of the embodiment of the present disclosure, terms such as first, second, A, B, (a), and (b) may be used. These terms are used merely to distinguish one component from other components, and the essentials, order, or sequence of the components are not limited by the terms.

Unless otherwise defined, all terms including technical and scientific terms used herein have the same meaning as commonly understood by one of ordinary skill in the art to which the present disclosure belongs. Terms that are generally defined in commonly used dictionaries should be construed as having meanings consistent with their contextual usage in the relevant technical field, and, unless explicitly defined in this application, and should not be construed in an idealized or unduly formal sense.

It will be understood that when a component is referred to as being “associated” with another component, it can be directly associated with or connected to the other component, but other intervening components may be present therebetween.

The terms used in the present disclosure are used only to describe a specific embodiment, and are not intended to limit the present disclosure. A singular expression includes a plural expression unless a description to the contrary is specifically pointed out in context. It will be further understood that the terms “comprise”, “include”, “have”, etc. when used in this specification, specify the presence of stated features, numbers, steps, operations, elements, or combinations thereof but do not preclude the possibility of the presence or addition of one or more other features, numbers, steps, operations, elements, or combinations thereof.

Hereinafter, embodiments of the present disclosure will be described in detail with reference to the attached drawings. In the description of the present disclosure, independent reference numerals are used to designate the same components in the drawings to facilitate overall understanding.

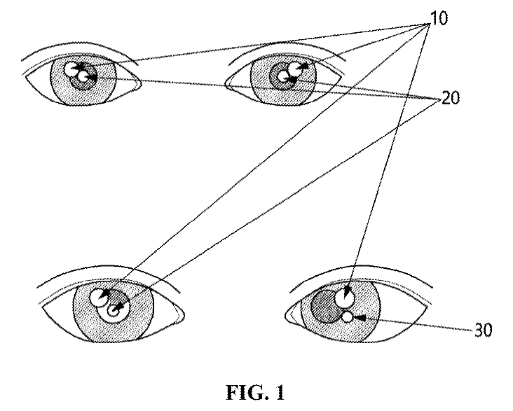

FIG. 1 is a diagram illustrating a situation in which an iris recognition error caused by pupil deviation of a non-dominant eye relative to a dominant eye occurs according to an embodiment of the present disclosure.

Referring to FIG. 1, illustrated is a process of removing specular reflections 10 generated by other light sources from iris images captured by an iris recognition apparatus 100 according to an embodiment of the present disclosure, and recognizing specular reflections 20 generated by an infrared light-emitting diode (IR-LED) used in the iris recognition apparatus 100, thus recognizing irises in a situation in which pupil deviation of a non-dominant eye occurs.

Here, it can be seen that the IR-LED used in the iris recognition apparatus 100 is positioned to face the center of each iris, and thus specular reflections 20 are generated.

However, when the right eye is a dominant eye, there may occur the case where a specular reflection 30 is not positioned at the center of the iris of the left eye which is a non-dominant eye, as the eyeball of the left eye turns toward the right eye, thus making it impossible to capture an iris image of the entire left eye.

On the other hand, when the left eye is a dominant eye, a phenomenon in which the eyeball of the right eye which is a non-dominant eye, turns toward the left eye may occur.

Due to the positional change of the eyeballs, a problem may arise in that accurate biometric information of both eyes cannot be obtained, and iris recognition may fail.

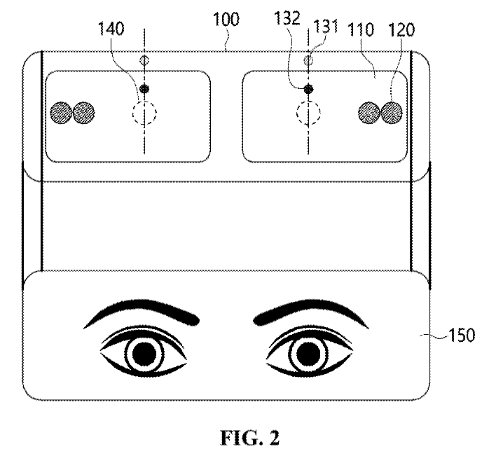

FIG. 2 is a diagram illustrating a goggle-type iris recognition apparatus according to an embodiment of the present disclosure.

Referring to FIG. 2, it can be seen that the goggle-type iris recognition apparatus according to the embodiment of the present disclosure is illustrated.

An iris recognition apparatus 100 for correcting pupil deviation attributable to a dominant eye may be applied to typical portable or standalone type devices without having a guide device, even in the case of non-goggle-type devices.

The iris recognition apparatus 100 for correcting pupil deviation attributable to a dominant eye may include a half mirror 110, an iris recognition IR-LED indicator 120, a camera module 130, additional indicators 131 and 132, and an eyeball position guidance unit 140.

The half mirror 110 may display a reflected image of a user’s pupils and allow infrared (IR) images to pass therethrough.

The half mirror 110 may guide the user to accurately align his or her eyes with iris recognition positions while checking the user’s pupils through the reflected image of the user’s pupils.

The user may align his or her eyes with designated iris recognition positions of the iris recognition apparatus100 while viewing his or her pupils reflected in the half mirror 110. By means of this, the gaze of the user may be guided to the accurate iris recognition positions for iris recognition.

The half mirror 110 may collect iris information from on images transmitted from an IR-LED or a camera.

The iris recognition IR-LED indicator 120 may provide infrared (IR) light sources required for iris recognition.

Here, the iris recognition IR-LED indicator 120 may create ideal conditions in which, when infrared light reaches the center of the user’s pupil and a specular reflection is reflected toward an iris recognition position that is the imaging center of the camera module 130, the iris recognition apparatus 100 for correcting pupil deviation attributable to a dominant eye may capture an iris image at an accurate iris recognition position.

The iris recognition apparatus 100 may accurately obtain data required for iris recognition due to the light reflection of IR-LED positioned to face the center of each pupil, thus providing highly reliable iris recognition.

Here, the iris recognition IR-LED indicator 120 may determine whether a specular reflection of iris recognition IR-LED is present within the pupil through a difference image by turning on/off the iris recognition IR-LED. A technique for recognizing pupils and irises is not included in the scope of the present disclosure, and may be implemented using conventional technologies.

The camera module 130 may capture iris images.

The additional indicators 131 and 132 may be arranged within 2 cm above the iris recognition position reflected in the half mirror 110.

Here, the additional indicator 131 may be a visible-light LED indicator.

Here, the additional indicator 132 may be an IR-LED indicator.

The IR-LED additional indicator 132 may be used on a rear surface of the half mirror 110. When the visible-light LED additional indicator 131 is mounted on the rear surface of the half mirror, the user cannot view the visible-light LED additional indicator 131.

The additional indicators 131 and 132 may recognize a dominant eye (leading eye), and may guide the user so that the center of the pupil of a non-dominant eye is positioned at the imaging center of the camera module 130 by turning on the non-dominant eye-side additional indicator.

The visible-light LED additional indicator 131 may be arranged ‘above’ the half mirror 110.

The IR-LED additional indicator 132 may be arranged on the ‘rear surface’ of the half mirror 110.

The IR-LED additional indicator 132 may be arranged within 2 cm from the iris recognition position reflected in the half mirror 110 captured by the camera module 130. For example, the IR-LED additional indicator 132 may be arranged within 2 cm ‘above’ the iris recognition position reflected in the half mirror 110.

When this range is exceeded, a specular reflection for the IR-LED additional indicator 132 may be generated on the iris of the user, thus obscuring the iris biometric information.

The eyeball position guidance unit 140 may indicate a region or a position with which the user aligns his or her eyes.

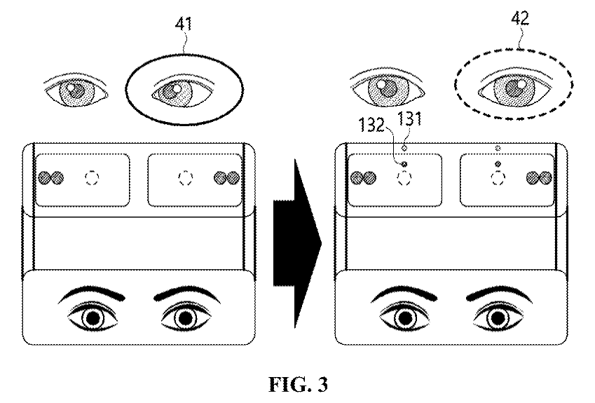

FIG. 3 is a diagram illustrating a dominant eye correction process according to an embodiment of the present disclosure.

Referring to FIG. 3, the iris recognition apparatus 100 for correcting pupil deviation attributable to a dominant eye according to the embodiment of the present disclosure may guide a user to naturally align his or her gaze with the right eye when the right eye is a dominant eye. Due thereto, the pupil of the left eye turns toward the right eye, and a problem may arise in that the iris recognition apparatus 100 for correcting pupil deviation attributable to a dominant eye cannot accurately capture the entire iris of the left eye through a camera (41). As a result, there is a risk in that iris information of the left eye may be incompletely collected, thereby increasing the likelihood of errors occurring during the iris recognition process.

For example, when the user whose right eye is a dominant eye aligns his or her gaze in front of the apparatus, only a portion of the iris image, rather than the entire iris image, is captured by the camera while the pupil 41 of the left eye turns to the right. In this way, the iris recognition apparatus 100 for correcting pupil deviation attributable to a dominant eye cannot accurately capture both iris images.

In order to solve this problem, the iris recognition apparatus 100 for correcting pupil deviation attributable to a dominant eye may provide a function of adjusting the left and right gaze directions of the user by utilizing the additional indicators (LEDs) 131 and 132.

The iris recognition apparatus 100 for correcting pupil deviation attributable to a dominant eye may activate the additional indicators 131 and 132 so that, when the user whose right eye is a dominant eye is positioned in front of the apparatus, the left eye of the user looks at an accurate position. The additional indicators 131 and 132 may momentarily guide the user to align his or her gaze with the accurate position, whereby it can be seen that the pupil of the left eye is adjusted to a correct direction (42).

In the state in which the additional indicators 131 and 132 of the iris recognition apparatus 100 for correcting pupil deviation attributable to a dominant eye are turned on, the user momentarily looks at the corresponding light sources (indicators). At this moment, when both eyes of the user become aligned, and the iris recognition apparatus 100 for correcting pupil deviation attributable to a dominant eye may simultaneously capture images of both irises at an accurate timing. The user briefly focuses his or her gaze on the bright light of the additional indicators 131 and 132, thus enabling both of the dominant eye and the non-dominant eye to be accurately aligned with the camera.

After adjusting the gaze of the user, the iris recognition apparatus 100 for correcting pupil deviation attributable to a dominant eye may automatically turn off the additional indicators when all of necessary iris images are captured.

In this way, even in the case of the user whose right eye is the dominant eye, images of both irises may be perfectly captured, whereby reliable iris recognition is enabled. Eye alignment using the additional indicators 131 and 132 enables stable and accurate iris recognition to be performed in various user environments, thereby improving the reliability of biometric recognition.

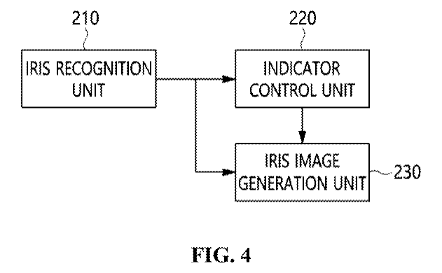

FIG. 4 is a block diagram illustrating an iris recognition apparatus for correcting pupil deviation attributable to a dominant eye according to an embodiment of the present disclosure.

Referring to FIG. 4, the iris recognition apparatus 100 for correcting pupil deviation attributable to a dominant eye according to an embodiment of the present disclosure may include an iris recognition unit 210, an indicator control unit 220, and an iris image generation unit 230.

The iris recognition unit 210 may recognize specular reflections generated at the face of a user, the pupil positions of both eyes, and iris recognition positions using the camera module 130.

Here, the iris recognition unit 210 may detect the face of the user and recognize the pupil positions of the left eye and the right eye of the user by utilizing the camera module 130.

For example, when the user is positioned in front of the apparatus, the iris recognition unit 210 may allow the camera to detect the face of the user and identify the positions of two pupils corresponding to the left eye and the right eye. By means of this process, the iris recognition unit 210 may perform initial settings for iris recognition.

The indicator control unit 220 may activate (turn on) the iris recognition IR-LED indicator 120.

Here, when the pupil positions are recognized by the iris recognition unit 210, the indicator control unit 220 may generate specular reflections at the iris recognition positions by activating (turning on) the iris recognition IR-LED indicator 120.

The iris recognition unit 210 may determine whether specular reflections of the left and right pupils are generated in the state in which the iris recognition IR-LED indicator 120 is turned on (activated).

In this case, when the center positions of the specular reflections deviate from the centers of the pupil positions by a preset threshold distance or longer, the iris recognition unit 210 may determine that the pupil positions and the positions of specular reflections are misaligned.

Here, the iris recognition unit 210 may recognize a pupil on which a specular reflection is generated by infrared light emitted from the corresponding IR-LED. Such a specular reflection may provide assistance in clearly identifying the positions of the pupils and iris images during an iris recognition process.

For example, the iris recognition unit 210 may determine whether specular reflections are correctly aligned with two pupils.

When a specular reflection is not detected from any one of both pupils, the indicator control unit 220 may turn on the additional indicators 131 and 132.

For example, when a specular reflection is not detected from the right pupil, the indicator control unit 220 may guide the user to accurately align his or her eyes by activating (turning on) the additional indicators 131 and 132 on the side of the right pupil (i.e., right-eye side). By means of this process, the gaze of the user may be adjusted to the accurate iris recognition positions.

The iris image generation unit 230 may capture iris images of both eyes when specular reflections are detected from both pupils.

The iris image generation unit 230 may store iris biometric information as iris images in the state in which the accurate positions of pupils are secured through the specular reflections generated by the iris recognition IR-LED indicator 120. Such iris images may be exploited as data for biometric authentication.

The indicator control unit 220 may deactivate (turn off) the additional indicators 131 and 132 after the iris images of both eyes have been captured.

The stored iris images may be registered and used to verify the identity of the user or authenticate the user.

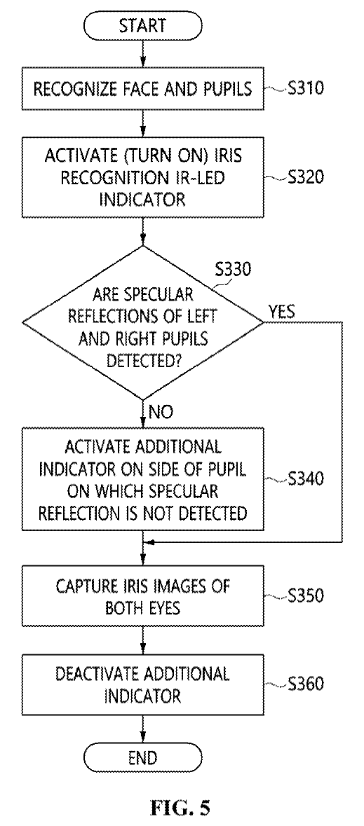

FIG. 5 is an operation flowchart illustrating an iris recognition method for correcting pupil deviation attributable to a dominant eye according to an embodiment of the present disclosure.

Referring to FIG. 5, the iris recognition method for correcting pupil deviation attributable to a dominant eye according to the embodiment of the present disclosure may recognize a face and pupils at step S310.

That is, at step S310, the face of a user may be detected and the pupil positions of the left eye and the right eye may be recognized by utilizing a camera module.

Here, at step S310, the pupil positions of both eyes may be guided to iris recognition positions by reflecting the pupils of the user using a half mirror.

Here, at step S310, the face of the user and the pupil positions of both eyes may be recognized, in which the face of the user may be recognized by reflecting the pupils of the user through the half mirror and then guiding the pupil positions of both eyes to the iris recognition positions using the camera module.

For example, at step S310, when the user is positioned in front of the apparatus, the camera may detect the face of the user and identify the positions of two pupils corresponding to the left eye and the right eye. By means of this process, at step S310, initial settings for iris recognition may be performed.

Further, the iris recognition method for correcting pupil deviation attributable to a dominant eye according to the embodiment of the present disclosure may activate (turn on) the iris recognition IR-LED indicator 120 at step S320.

Here, at step S320, when the pupil positions are recognized at step S310, specular reflections may be generated at the iris recognition positions by activating (turning on) the iris recognition IR-LED indicator 120.

In addition, the iris recognition method for correcting pupil deviation attributable to a dominant eye according to the embodiment of the present disclosure may detect specular reflections of left and right pupils at step S330.

That is, at step S330, whether specular reflections of the left and right pupils are generated may be determined in the state in which the iris recognition IR-LED indicator 120 is turned on (activated).

Here, at step S330, whether the pupil positions are aligned with the positions of specular reflections may be determined.

In this case, at step S330, when the center position of each of the specular reflections deviates from the center of the corresponding pupil position by a preset threshold distance or longer, it may be determined that the pupil position and the position of the corresponding specular reflection are misaligned.

Here, at step S330, a pupil on which a specular reflection is generated by infrared light emitted from the corresponding IR-LED may be recognized. Such a specular reflection may provide assistance in clearly identifying the positions of the pupils and iris images during an iris recognition process.

For example, at step S330, whether specular reflections are correctly aligned with two pupils may be determined.

Furthermore, the iris recognition method for correcting pupil deviation attributable to a dominant eye according to the embodiment of the present disclosure may activate (turn on) additional indicators 131 and 132 on the side of a pupil on which a specular reflection is not detected at step S340.

That is, at step S340, when a specular reflection is not detected on any one of both pupils at step S330, the additional indicators 131 and 132 may be activated (turned on).

For example, at step S340, when a specular reflection is not detected from the right pupil, the user may be guided to accurately align his or her eye by activating (turning on) the additional indicators 131 and 132 on the side of the right pupil. By means of this process, step S340 may be performed to adjust the gaze (direction) of the user to the accurate iris recognition position.

Furthermore, the iris recognition method for correcting pupil deviation attributable to a dominant eye according to the embodiment of the present disclosure may capture iris images of both eyes at step S350.

That is, at step S350, when specular reflections are detected on both pupils at step S330 or S340, the iris images of both eyes may be captured.

Here, at step S350, iris biometric information may be stored as iris images in the state in which the accurate positions of pupils are secured through the specular reflections generated by the iris recognition IR-LED indicator 120. Such iris images may be exploited as data for biometric authentication.

Furthermore, the iris recognition method for correcting pupil deviation attributable to a dominant eye according to the embodiment of the present disclosure may deactivate (turn off) the additional indicators at step S360.

That is, at step S360, the additional indicators 131 and 132 may be deactivated (turned off) after the iris images of both eyes have been captured.

The stored iris images may be registered and used to verify the identity of the user or authenticate the user.

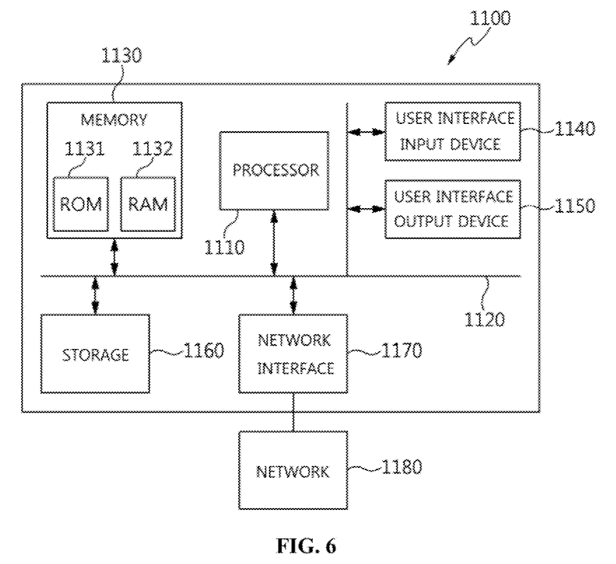

FIG. 6 is a diagram illustrating a computer system according to an embodiment of the present disclosure.

Referring to FIG. 6, an iris recognition apparatus 100 for correcting pupil deviation attributable to a dominant eye according to an embodiment of the present disclosure may be implemented in a computer system 1100 such as a computer-readable storage medium. As illustrated in FIG. 6, the computer system 1100 may include one or more processors 1110, memory 1130, a user interface input device 1140, a user interface output device 1150, and storage 1160, which communicate with each other through a bus 1120. The computer system 1100 may further include a network interface 1170 connected to a network 1180. Each processor 1110 may be a Central Processing Unit (CPU) or a semiconductor device for executing processing instructions stored in the memory 1130 or the storage 1160. Each of the memory 1130 and the storage 1160 may be any of various types of volatile or nonvolatile storage media. For example, the memory 1130 may include Read-Only Memory (ROM) 1131 or Random Access Memory (RAM) 1132.

The present disclosure may solve a phenomenon in which an iris image of a non-dominant eye is distorted due to pupil deviation (displacement) attributable to dominance of a dominant eye (leading eye).

Further, the present disclosure may capture accurate images of an iris at all angles by solving a phenomenon in which a specular reflection does not occur at an accurate iris recognition position due to pupil deviation (displacement).

As described above, in the iris recognition apparatus and method for correcting pupil deviation attributable to a dominant eye according to the present disclosure, the configurations and schemes in the above-described embodiments are not limitedly applied, and some or all of the above embodiments can be selectively combined and configured so that various modifications are possible.

What is claimed is:

1. An iris recognition apparatus for correcting pupil deviation attributable to a dominant eye, comprising:
an iris recognition unit configured to recognize specular reflections generated on a face of a user, pupil positions of both eyes, and iris recognition positions using a camera module, and to determine whether each of the pupil positions is aligned with a position of a corresponding specular reflection;
an indicator control unit configured to generate the specular reflections at the iris recognition positions by activating an iris recognition infrared light-emitting diode (IR-LED) indicator, and to activate an additional indicator arranged on a side of an eyeball on which a pupil position is misaligned with a position of a specular reflection; and
an iris image generation unit configured to capture iris images of both of the eyes in a state in which the specular reflections are aligned with the pupil positions of both of the eyes.

2. The iris recognition apparatus of claim 1, wherein the iris recognition unit is configured to, when a center position of the corresponding specular reflection deviates from a center of the corresponding pupil position by a preset threshold distance or longer, determine that the pupil position is misaligned with the position of the corresponding specular reflection.

3. The iris recognition apparatus of claim 1, further comprising:
a half mirror configured to guide the pupil positions of both of the eyes to the iris recognition positions by reflecting pupils of the user.

4. The iris recognition apparatus of claim 3, wherein the additional indicator is arranged within 2 cm from each iris recognition position reflected in the half mirror.

5. The iris recognition apparatus of claim 4, wherein:
the additional indicator comprises a visible-light LED additional indicator and an IR-LED additional indicator,
the visible-light LED additional indicator is arranged above the half mirror, and
the IR-LED additional indicator is arranged on a rear surface of the half mirror.

6. An iris recognition method for correcting pupil deviation attributable to a dominant eye, performed by an iris recognition apparatus for correcting pupil deviation attributable to a dominant eye, the iris recognition method comprising:
recognizing a face of a user and pupil positions of both eyes using a camera module;
generating specular reflections at iris recognition positions by activating an iris recognition IR-LED indicator;
recognizing the specular reflections generated at the iris recognition positions;
determining whether each of the pupil positions is aligned with a position of a corresponding specular reflection;
when it is determined that each of the pupil positions is misaligned with the position of the corresponding specular reflection, activating an additional indicator arranged on a side of an eyeball on which the pupil position is misaligned with the position of the corresponding specular reflection; and
capturing iris images of both of the eyes in a state in which the specular reflections are aligned with the pupil positions of both of the eyes.

7. The iris recognition method of claim 6, wherein the determining comprises:
when a center position of the corresponding specular reflection deviates from a center of the corresponding pupil position by a preset threshold distance or longer, determining that the pupil position is misaligned with the position of the corresponding specular reflection.

8. The iris recognition method of claim 6, wherein the recognizing comprises:
guiding the pupil positions of both of the eyes to the iris recognition positions by reflecting pupils of the user by using a half mirror.

9. The iris recognition method of claim 8, wherein the additional indicator is arranged within 2 cm from each iris recognition position reflected in the half mirror.

10. The iris recognition method of claim 9, wherein:
the additional indicator comprises a visible-light LED additional indicator and an IR-LED additional indicator,
the visible-light LED additional indicator is arranged above the half mirror, and
the IR-LED additional indicator is arranged on a rear surface of the half mirror.
